(19) United States
(12) Patent Application Publication
(10) Pub. No.: US 2013/0096665 A1
(43) Pub. Date: Apr. 18, 2013

(54) VASCULAR GRAFT HAVING LIMITED END STRUCTURE

(71) Applicant: TriVascular, Inc., Santa Rosa, CA (US)

(72) Inventor: Michael V. Chobotov, Santa Rosa, CA (US)

(73) Assignee: TriVascular, Inc., Santa Rosa, CA (US)

(21) Appl. No.: 13/650,705

(22) Filed: Oct. 12, 2012

Related U.S. Application Data
(60) Provisional application No. 61/547,470, filed on Oct. 14, 2011.

(51) Int. Cl. A61F 2/06; B23P 17/04

(52) U.S. Cl. 623/1.13; 29/428

(57) ABSTRACT

The vascular graft is located within a lumen of a vessel. The vascular graft includes a tubular structure and an attachment ring connected thereto. A stent attachment structure is connected to the attachment ring. The stent attachment structure is connected to an inner surface of the vessel to secure the location of the vascular graft within the lumen. The vascular graft is made according to a method in which the end of the tubular structure is folded over the attachment ring. Alternatively, the vascular graft has circumferential slits near the end thereof. Strap structures extend through the slits and are folded for connection to the stent attachment structure. The vascular graft is made according to a method in which the strap structures are inserted through the slits and folded for connection to the stent attachment structure.

### CROSS-REFERENCE TO RELATED APPLICATION

This application claims the benefit of U.S. Provisional Application No. 61/547,470, filed Oct. 14, 2011, the entire contents of which are incorporated by reference herein.

### FIELD OF THE INVENTION

The present invention relates generally to a vascular graft and, more specifically, to a vascular graft having a limited end structure to facilitate securing the vascular graft within a vessel.

### BACKGROUND OF THE INVENTION

An aneurysm is a portion of a blood vessel which has been abnormally dilated to have, for example, the shape of a balloon or a bulge. The dilation of the blood vessel associated with the aneurysm is typically weaker than the healthy blood vessel. It is possible for a blood vessel to be sufficiently weakened by an aneurysm to pose a risk of the blood vessel rupturing at the site of the aneurysm. Such a rupture normally results in internal hemorrhaging which is typically very serious.

An aneurysm in a blood vessel may be treated by inserting a tubular structure of a vascular graft into the blood vessel such that the inlet of the tubular structure is upstream of the aneurysm and the outlet of the tubular structure is downstream of the aneurysm. Consequently, the blood flows within the tubular structure through the region of the blood vessel in which the aneurysm is located. The containment of the blood flow within the tubular structure shields the aneurysm from direct contact with the blood flow and corresponding pressure thereof within the blood vessel. The shielding of the aneurysm from the direct contact with the blood flow reduces the outward radial force which would otherwise be produced by the fluid pressure of the blood within the blood vessel. Consequently, the possibility of the aneurysm rupturing and associated loss of blood is reduced.

The vascular graft may have an annular cuff or sealing member, such as a sealing ring, secured to the outer surface of the tubular structure adjacent to the upstream end thereof. The cuff or sealing ring engages the inner surface of the blood vessel to provide a seal which obstructs leakage of blood at the inlet of the tubular structure between the outer surface thereof and inner surface of the blood vessel which if present, may enter the region of the vessel between the tubular structure and aneurysm and bypass the tubular structure. The bypass of the tubular structure diminishes the advantages thereof and, consequently, is preferably prevented.

An aneurysm may be present in a section of a primary vessel of the blood vessel which is adjacent to one or more branch vessels which intersect the primary vessel. Such a blood vessel may have a blood flow therein which is directed from the primary vessel upstream of the branch vessels toward and into the section of the primary vessel which is intersected by the branch vessels. The blood which enters this section of the primary vessel may be diverted, in part, into the one or more branch vessels or may be combined with blood which enters this section of the primary vessel from the branch vessels. In either case, blood flows from the section of the primary vessel which is intersected by the branch vessels into the section of the primary vessel which has the aneurysm.

The aneurysm may be treated by locating a vascular graft in the primary vessel such that the inlet of the tubular structure is located upstream of the aneurysm and downstream of the downstream end of the intersection of the primary vessel by the branch vessels arteries. The location of the inlet of the tubular structure downstream of the downstream end of the intersection of the primary vessel by the branch vessels substantially eliminates possible interference by the tubular structure with the blood flow between the primary vessel and branch vessels.

The vascular graft may have an annular cuff or sealing ring secured to the outer surface of the tubular structure to obstruct leakage of blood at the inlet of the tubular structure between the outer surface thereof and inner surface of the blood vessel. In order for the cuff or sealing ring to provide an adequate seal with the inner surface of the blood vessel, the section of the blood vessel between the downstream end of the branch vessels and aneurysm is required to have a sufficient longitudinal dimension to provide adequate surface area for the cuff or sealing ring to engage.

The vascular graft typically includes stent attachment structure which is connected to the tubular structure for securing the location of the vascular graft within the blood vessel. The stent attachment structure may be connected to the tubular structure between the upstream end thereof and the cuff or sealing ring, and require a sufficient longitudinal dimension of the tubular structure for the connection of the stent attachment structure thereto. The required longitudinal dimension of the tubular structure for the connection of the stent attachment structure thereto may be significant and result in the longitudinal dimension of the tubular structure from the upstream end thereof to the downstream end of the cuff or sealing ring being substantial. Consequently, the use of the vascular graft for the treatment of an aneurysm in a primary vessel having one or more branch vessels upstream of the aneurysm may be limited to a primary vessel which has a sufficient longitudinal dimension between the downstream end of the branch vessels and the aneurysm, such as 10 to 15 millimeters (mm.). The sufficient longitudinal dimension of the primary vessel between the downstream end of the one or more branch vessels and aneurysm is required to provide for the inlet end of the tubular structure to be downstream of the downstream end of the intersection between the one or more branch vessels and primary vessel. Also, the sufficient longitudinal dimension of the primary vessel between the one or more branch vessels and aneurysm is required to provide a surface against which the cuff or sealing ring is engaged to provide the seal. Consequently, the required longitudinal dimension of the primary vessel between the aneurysm and the downstream end of the intersection of the primary vessel by the one or more branch vessels may limit the use of the vascular graft to patients which have the required longitudinal dimension in the primary vessel. As a result, the use of the vascular graft to treat an aneurysm in a primary vessel which is downstream of the intersection thereof by one or more branch vessels may be limited.

An example of a blood vessel which includes a primary vessel intersected by more than one branch vessel is the intersection of the aorta by the renal arteries in a human. The section of the aorta to which blood flows from the region of the aorta intersected by the renal arteries may, unfortunately, have an aneurysm. An aneurysm in the region of the aorta which is downstream of the intersection thereof by the renal arteries may be treated by inserting a vascular graft in the aorta.

### SUMMARY OF THE INVENTION

The vascular graft of the present invention is located within a lumen of a vessel. The vascular graft includes a tubular structure having an end structure. An attachment ring is connected to the end structure. The attachment ring is contained in a ring plane which is perpendicular to the end structure. A stent attachment structure is connected to the attachment ring. The stent attachment structure has an anchor structure for connection to an inner surface of the vessel to secure the location of the vascular graft within the lumen. A method of making the vascular graft includes locating the attachment ring around the outer surface of the tubular structure adjacent to an end thereof, and folding the end outward and backward onto the outer surface of the tubular structure to secure the attachment ring between outer and inner layers of the tubular structure.

An alternative embodiment of the vascular graft of the present invention is located within the lumen of the vessel. The vascular graft includes a tubular structure having an end structure. The tubular structure has one or more circumferential slits formed in the end structure. The vascular graft has one or more strap structures each of which extends through a respective one of the one or more circumferential slits and is folded such that the one or more strap structures each has an outer leg structure and an inner leg structure. A stent attachment structure has one or more stent structures each of which is connected to the outer leg structure and inner leg structure of a respective one of the one or more strap structures. The one or more stent structures each has an anchor structure for connection to an inner surface of the vessel to secure the location of the vascular graft within the lumen. A method of making the vascular graft includes inserting the one or more strap structures through respective ones of the circumferential slits. Following the insertion, the one or more strap structures are folded such that each of the strap structures has outer and inner leg structures. The outer and inner leg structures are then connected to the stent attachment structure.

The attachment ring, and one or more strap structures provide for the connection of the stent attachment structure to the end structure of the tubular structure. The attachment ring and one or more strap structures further provide for the end structure to have a reduced longitudinal dimension. Consequently, the vascular graft may be used to treat an aneurysm which is located in a primary vessel of a vessel downstream of one or more branch vessels of the vessel where the longitudinal dimension between the aneurysm and the downstream end of the intersection of the primary vessel by the one or more branch vessels is reduced, such as to 5 mm. The reduction in the required longitudinal dimension of the primary vessel between the aneurysm and the downstream end of the intersection of the primary vessel by the one or more branch vessels increases the types of aneurysms and, more specifically, the range of locations thereof in a primary vessel relative to the one or more branch vessels which intersect the primary vessel which may be treated by the vascular graft. Consequently, the range of patients who may be treated by the vascular graft is expanded.

These and other features of the invention will be more fully understood from the following description of specific embodiments of the invention taken together with the accompanying drawings.

### BRIEF DESCRIPTION OF THE DRAWINGS

In the drawings:

FIG. 1 is a cross-sectional view of the vascular graft of the present invention, the vascular graft being shown as secured within a vessel adjacent to an aneurysm therein;

FIG. 2 is an enlarged view of the circled portion to of the vascular graft of FIG. 1, the vascular graft being shown as having a tubular structure to which is connected an attachment ring, the attachment ring being shown as having a stent attachment structure connected thereto;

FIG. 3 is an enlarged perspective view of the attachment ring of FIG. 2, the attachment ring being shown as having a ring structure and multiple loop structures integrally connected thereto;

FIGS. 4A to 4C are plan views of the attachment ring of FIG. 3, the attachment ring being shown as having various configurations resulting from incremental folding thereof;

FIG. 5 is an elevational view of an alternative embodiment of the vascular graft of FIG. 1, the vascular graft being shown as having a tubular structure including circumferential slits through which strap structures extend for connection to the stent attachment structure;

FIG. 6 is an enlarged view of the portion of the tubular structure and strap structure contained within the circled portion 6 of FIG. 5, the strap structure being shown as inserted through the circumferential slit and folded; and

FIG. 7 is an elevational view of one of the strap structures of FIG. 5, the strap structure being shown before the insertion thereof through the circumferential slit, the strap structure being further shown before the folding thereof which follows the insertion of the strap structure through the circumferential slit.

Corresponding reference characters indicate corresponding parts throughout the several views of the drawings.

### DETAILED DESCRIPTION OF THE INVENTION

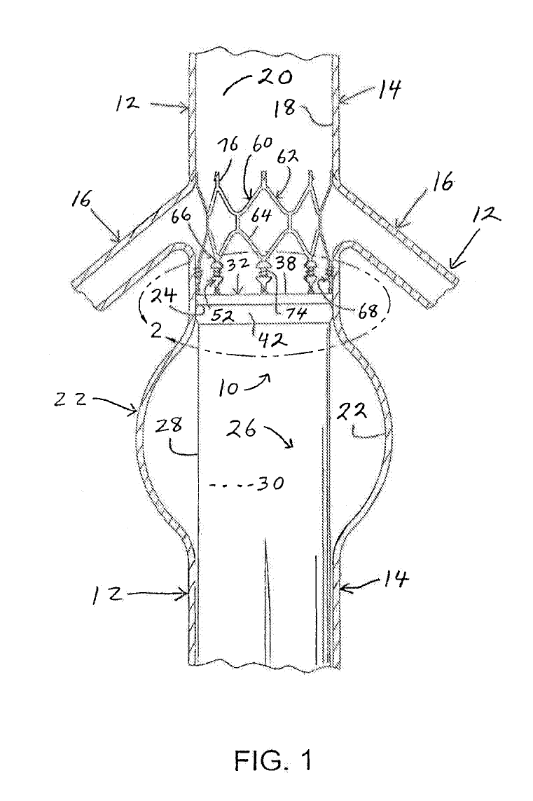

Referring to the drawings and more specifically to FIG. 1, the vascular graft 10 is located within a vessel 12 which has a primary vessel 14 and a pair of branch vessels 16 which extend laterally from the primary vessel. The primary vessel 14 has an inner surface 18 which defines a lumen 20. The primary vessel 14 is shown as having an aneurysm 22 located below the branch vessels 16. The aneurysm 22 is located a sufficient distance below the branch vessels 16 such that a section of the primary vessel 14 which is not dilated is located between the branch vessels 16 and aneurysm 22. The section of the primary vessel 14 between the branch vessels 16 and aneurysm 22 defines a landing zone 24.

The vascular graft 10 has a tubular structure 26 which includes outer and inner surfaces 28, 30. The tubular structure 26 may be formed of expanded polytetrafluoroethylene (ePTFE) or polyurethane. The tubular structure 26 may be formed of biocompatible materials, such as biocompatible polymers including those which are known. Such polymers may include fillers such as metals, carbon fibers, glass fibers or ceramics. Also, such polymers may include olefin polymers, polyethylene, polypropylene, polyvinyl chloride, polytetrafluoroethylene which is not expanded, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, copolymers, and combinations thereof. Also, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, and natural silk may be included in the tubular structure 26.

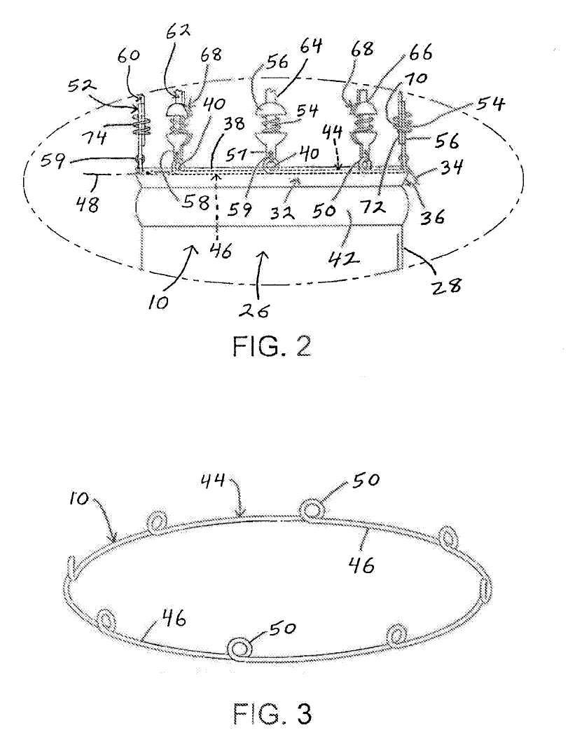

The upper longitudinal portion of the tubular structure 26 including the end thereof defines an end structure 32, as shown in FIGS. 1 and 2. The upper end portion of the tubular structure 26 is folded outward and back onto itself such that an outer layer 34 of the end structure 32 overlays an inner layer 36 thereof. An end surface 38 is thereby defined by the integral junction of the outer and inner layers 34, 36. The end surface 38 has a generally transverse relation to the tubular structure 26.

The end structure 32 includes circumferential slits 40 which are formed in the end surface 38 such that the spacing between adjacent pairs of the slits is substantially the same. The slits 40 are substantially parallel to the outer and inner surfaces 28, 30.

The vascular graft 10 includes an inflatable cuff or sealing ring structure 42 which is secured to the end structure 32, as shown in FIGS. 1 and 2. The cuff or sealing ring structure 42 has a longitudinal location relative to the tubular structure 26 such that the outer layer 34 is located between the cuff or sealing ring structure and end surface 38.

The longitudinal portion of the end structure 32 between the cuff or sealing ring structure 42 and end surface 38 has a cylindrical configuration. An alternative embodiment of the end structure 38 provides for the longitudinal section thereof between the cuff or sealing ring structure 42 and end surface 38 to have a diameter which increases in the longitudinal direction relative to the tubular structure 26 from the cuff or sealing ring structure to the end surface. Consequently, the longitudinal section of the end structure 38 between the cuff or sealing ring structure 42 and end surface 38 is tapered which provides improved hemodynamics.

The vascular graft 10 has an attachment ring 44 which includes a ring structure 46 which is contained in a ring plane 48. The attachment ring 44 has loop structures 50 which are integrally connected to the ring structure 46, as shown in FIG. 3. The loop structures 50 extend from the ring structure 46 in substantially the same direction relative thereto. The loop structures 50 are substantially perpendicular to the ring plane 48. The number of loop structures 50 corresponds to number of slits 40 in the end surface 38 and the spacing between the loop structures corresponds to the spacing between the slits.

The attachment ring 44 is formed by a single turn of wire. An embodiment of the wire of the attachment ring 44 is formed of nitinol material and has a cross-section the diameter of which is from about 0.005 inches (in.) to about 0.02 inches (in.). In some embodiments, the diameter of the cross-section is 0.010 inches (in.). The wire of the attachment ring 44 is shape set on a tool to retain the configuration of the attachment ring for facilitating the connection and integration thereof into the tubular structure 26. An alternative preferred embodiment of the attachment ring 44 is formed from several turns of a wire, such as 3 to 5 turns of a wire formed of nitinol material and having a cross-section the diameter of which is 0.004 in. to 0.008 in. A further alternative preferred embodiment of the attachment ring 44 is formed from 6 to 8 turns of preformed wire of nitionol material which is formed on a mandrel such that the turns define a coil in which the sides of the turns directly contact one another.

The attachment ring 44 is connected to the end structure 32 by locating ring structure 46 around the tubular structure 26 in transverse relation thereto, as shown in FIGS. 1 and 2. The location of the ring structure 46 around the tubular structure 26 further provides for the loop structures 50 to extend from the ring structure in a proximal direction relative to the tubular structure 26 and for the loop structures to be located adjacent to respective slits 40 in the end surface 38. The longitudinal section of the tubular structure 26 which is between the slits 40 and the upper end of the tubular structure is folded outward and back onto the outer surface 28, as shown in FIG. 2. The longitudinal section of the tubular structure 26 which is folded back onto the outer surface 28 defines the outer layer 34 of the end structure 32. The longitudinal section of the tubular structure 26 on which the outer layer 34 is folded defines the inner layer 36 of the end structure 32. The folding of the outer layer 34 covers the section of the outer surface 28 which was provided by the inner layer 36 prior to the folding. Consequently, following the completion of the folding, the outer surface 28 of the section of the tubular structure 26 which is defined by the outer and inner layers 34, 36 is defined by the outer surface of the outer layer 34. Also, following the folding, the inner surface 30 of the section of the tubular structure 26 which is defined by the outer and inner layers 34, 36 is defined by the inner surface of the inner layer 36.

The folding of the outer layer 34 provides for sufficient longitudinal displacement thereof relative to the tubular structure 26 such that the integral junction between the outer layer and inner layer 36 is displaced in a distal direction relative to the tubular structure. Consequently, the loop structures 50 extend through the respective slits 40, as shown in FIG. 2. Following the completion of the folding of the outer layer 34 against the inner layer 36, the outer layer is secured to the inner layer, such as by welding. An embodiment of the end structure 32 provides for the outer layer 34 to have a longitudinal dimension of 2 mm. to 4 mm. relative to the tubular structure 26.

The securing of the outer layer 34 to the inner layer 36 following the folding shown in FIG. 2 connects the attachment ring 44 to the end structure 32 by securing the ring structure 46 between the outer and inner layers 34, 36. The connection of the attachment ring 44 to the end structure 32 provides for the ring plane 48 to be perpendicular to the end structure 32. Also, the connection provides for the loop structures 50 to extend from the ring structure 46 in the proximal direction relative to the tubular structure 26.

An alternative embodiment of the end structure 32 provides for formation of the slits 40 therein after the initiation of the folding of the outer layer 34. The slits 40 are formed during the folding by piercing the sections of the junction which face the loop structures 50 as the integral junction between the outer and inner layers 34, 36 approaches the loop structures. Following the piercing, further longitudinal displacement of the outer layer 34 in the distal direction relative to the tubular structure 26 results in the loop structures 50 extending through the slits 40.

The vascular graft 10 includes dogbone structures 52 each of which has an elongate central member 54. Each of the dogbone structures 52 has a pair of enlarged ear members 56 which extend from respective ends of the corresponding central member 54 to provide each dogbone structure 52 with the typical shape of a bone which is provided to dogs for chewing. Each of the dogbone structures 52 has an elongate tab member 57 which extends from one of the ear members 56 in a direction which is substantially parallel to the corresponding central member 54, as shown in FIG. 2. An eyelet 58 is located in each of the tab members 57.

The vascular graft 10 includes coil structures 59 each of which is defined by a wire which extends through a respective eyelet 58 and corresponding loop structure 50 to form a helical coil having multiple windings. Consequently, each of the coil structures 59 connects the dogbone structures 52 to the respective loop structures 50, as shown in FIG. 2. The connections of the dogbone structures 52 to the loop structures 50 provide for the central members 54 to extend from the respective loop structures 50 and attachment ring 54 in the proximal direction relative to the tubular structure 26.

The vascular graft 10 has a stent attachment structure 60 which includes a frame structure 62. The frame structure 62 has elongate stent structures 64 which are connected together in a cylindrical configuration which has a distal end 66.

The stent attachment structure 60 includes dogbone structures 68 each of which has an elongate central member 70 the ends of which terminate at enlarged ear member 72. The configuration of each of the dogbone structure 68 is substantially the same as the configuration of the dogbone structures 52. The dogbone structures 68 are connected to the distal end 66 of the frame structure 62 such that the central member 70 extends in the distal direction relative to the frame structure 62. The dogbone structures 68 correspond in size to the portions of the dogbone structures 52 which include the respective central members 54 and ear members 56.

The location of the dogbone structure 68 on the distal end 66 of the frame structure 62 provides for each of the dogbone structure 68 to adjoin a corresponding dogbone structure 52 of the attachment ring 44 when the distal end 66 of the frame structure 62 is positioned adjacent to the attachment ring 44 for connection thereto. The adjoining locations, and corresponding configurations and sizes of the dogbone structures 52, 58 provide for the adjoining pairs thereof to be superimposed relative to one another, as shown in FIGS. 1 and 2. The dogbone structures 52 are located outward of the dogbone structures 58 in the radial direction relative to the tubular structure 26.

A coil structure 74 including a wire which is wound around each pair of adjoining central members 54, 70 to secure the adjoining dogbone structures 52, 68 together. The coil structures 74 are each defined by shape set loops which are wound from wire formed of nitinol material having a cross-section the diameter of which is 0.005 in. to 0.008 in. The ear members 56, 72 obstruct relative longitudinal displacement between the adjoining dogbone structures 52, 68 since such displacement results in the coil structure 74 engaging the ear members. The connections between the respective pairs of the dogbone structures 52, 68 by the coil structure 74 provide for the connection of the attachment ring 44 to the stent attachment structure 60. The stent attachment structure 60 extends from the attachment ring 44 in the proximal direction relative to the tubular structure 26.

Alternative embodiments of the stent attachment structure 60 provide for the connection thereof to the attachment ring 44 before the connection of the attachment ring to the tubular structure 26.

An alternative embodiment of the vascular graft 10 provides for the dogbone structures 25 to be connected to the loop structures 50 by the respective coil structures 59 before the attachment ring 44 is connected to the tubular structure 26. A further alternative embodiment of the vascular graft 10 provides for the dogbone structures 25 to be connected to the attachment ring 44 by the insertion thereof through the eyelets 58 in a manner which is similar to the placement of keys on a keyring. Consequently, the coil structures 59 are not required when the attachment ring 44 is inserted through the eyelets 58.

An alternative embodiment of the vascular graft 10 provides for the distal end 66 of the frame structure 62 to have eyelets which are connected to the loop structures 50 by a coil structure which is comparable to the coil structure 59. A further alternative embodiment of the vascular graft 10 which includes the frame structure 62 the distal end 66 of which has eyelets provides for the insertion of the attachment ring 44 through the eyelets in a manner which is similar to the placement of keys on a keyring. Consequently, the coil structures 59 are not required when the attachment ring 44 is inserted through the eyelets in the distal end 66. Also, the dogbone structures 68 are not required when the attachment ring 44 is inserted through the eyelets in the distal end 66 or when the attachment ring 44 is connected to the loop structures 50 by the coil structure.

An alternative embodiment of the attachment ring 44 provides for the formation thereof from a fiber or plastic, such as string formed from polytetrafluoroethylene (PTFE). Forming the attachment ring 44 from fiber or plastic provides load distribution to the circumference of the end structure 32 for sutures or other discrete attachment points. The load distribution may also result from the connection of the stent attachment structure 60 to the alternative embodiment of the attachment ring 44. A further alternative embodiment of the attachment ring 44 provides for the integral formation thereof in the end structure 32 by rolling the tubular structure 26 in the longitudinal direction relative thereto to produce a ring-shaped reinforced portion in the end structure.

Alternative embodiments of the attachment ring 44 have wire, thread, or fibers wound therein such that the wire, thread, or fibers extend from the attachment ring proximally through the end surface 38. The wire, thread, or fibers which extend through the end surface 38 have terminations for connection to the stent attachment structure 60.

The stent attachment structure 60 includes anchor structures 76 which are formed in the proximal end of the frame structure 62. The anchor structures 76 provide connection of the stent attachment structure 60 to the inner surface 18 of the primary vessel 14 near the upstream intersections of the branch vessels 16 with the primary vessel 14. The connections of the anchor structures 76 to the inner surface 18 secures the location of the vascular graft 10 within the lumen 20 of the primary vessel 14. Also, the outward location of the dogbone structures 52 relative to the dogbone structures 58 results in frame structure 62 forcing the dogbone structures 52 and the associated attachment ring 44 and tubular structure 26 radially outward into engagement with inner surface 18 of the primary vessel 14.

The connection of the attachment ring 44 to the tubular structure 26 provided by the ring structure 46 allows for a reduced longitudinal dimension of the section of the tubular structure between the end surface 38 and cuff or sealing ring structure 42. Consequently, the vascular graft 10 can be used for the treatment of an aneurysm 22 which is located downstream of and relatively close to the branch vessels 16, as shown in FIG. 1. More specifically, the primary vessel 14 of the vessel 12 has an aneurysm 22 that is located relatively close to the branch vessels 16. Consequently, the primary vessel 14 has a landing zone 24 which has a reduced longitudinal dimension. The reduced longitudinal dimension of the tubular structure 26 between the end surface 38 and cuff or sealing ring structure 42 allows the location of the cuff or sealing ring structure 42 within the landing zone 24 and the end surface 38 to have a distal location relative to the downstream end of the intersection of the branch vessels 16 with the primary vessel 14. Consequently, the end surface 38 does not significantly interfere with the fluid flow between the primary vessel 14 and branch vessels 16. Also, the cuff or sealing ring structure 42 engages the inner surface 18 within the landing zone 24 to facilitate the seal between the cuff or sealing ring structure and inner surface 18 since the landing zone is not significantly dilated relative to the section of the primary vessel 14 in which the aneurysm 22 is located. As a result, the reduced longitudinal dimension of the tubular structure 26 between the end surface 38 and cuff or sealing ring structure 42 facilitates the treatment of the aneurysm 22 in the vessel 12.

The reduced longitudinal dimension of the section of the tubular structure 26 between the end surface 38 and cuff or sealing ring structure 42 is provided by the required longitudinal separation between the cuff or sealing ring structure and ring structure 46 being limited to the overlap between the outer and inner layers 34, 36. An alternative embodiment of the tubular structure 26 provides for the reduction of the longitudinal dimension of the section of the tubular structure 26 between the end surface 38 and cuff or sealing ring structure 42 by reducing the longitudinal separation between the cuff or sealing ring structure and ring structure 46. The longitudinal separation between the cuff or sealing ring structure 42 and ring structure 46 is reduced by the proximal end of the cuff or sealing ring structure being secured to the tubular structure 26 by a weld which also secures together the outer and inner layers 34, 36. The weld which secures the proximal end of the cuff or sealing ring structure 42 to the tubular structure 26 may be referred to as the proximal cuff or sealing ring proximal weld. The weld which secures together the outer and inner layers 34, 36 may be referred to as the ring encapsulation weld.

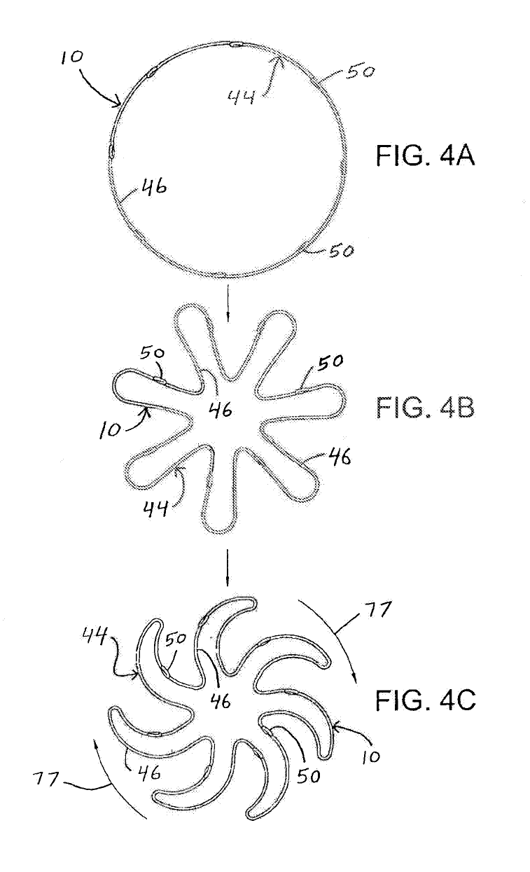

The attachment ring 44 can be folded incrementally as shown in FIGS. 4A to 4C. The attachment ring 44 before the folding is shown in FIG. 4A. A configuration of the attachment ring 44 following the initiation of the folding thereof is shown in FIG. 4B. A configuration of the attachment ring 44 following further folding thereof is shown in FIG. 4C. The attachment ring 44 shown in FIG. 4C is folded further in the directions 77 to move the arcuate portions of the ring structure 46 closer together. The folding of the attachment ring 44 facilitates reduction of the profile of the end structure 32 when the attachment ring is connected thereto. Reduction of the profile of the end structure 32 assists the insertion of the tubular structure 26 into the vessel 12 and displacement therein to locate the vascular graft 10 in the vessel. Also, the folding of the attachment ring 44 provides for the storage thereof for loading.

The lateral bending force required to fold the attachment ring 44 formed by a single turn of wire, such as is shown in FIGS. 4A to 4C and according to other methods of folding, is increased relative to the lateral bending force required to fold the alternative embodiment of the attachment ring 44 formed by several turns of wire where the materials of the wire are the same, and the combined cross-sectional dimensions of the several turns of wire are comparable to the cross-sectional dimension of the single turn of wire. Consequently, the alternative embodiment of the attachment ring 44 formed by several turns of wire has increased lateral flexibility which facilitates loading of the attachment ring and associated vascular graft into a cathether. The strength of the alternative embodiment of the attachment ring 44 formed by several turns of wire is comparable to the strength of the attachment ring 44 formed by a single turn of wire where the materials of the wire are the same, and the cross-sectional dimension of the single turn of wire is comparable to the combined cross-sectional dimensions of the several turns of wire. An example of the different lateral flexibilities is indicated by the lateral bending force required to fold the alternative embodiment of the attachment ring 44 formed from nitinol wire having 3 to 5 turns each turn of which has a diameter from 0.004 in. to 0.005 in. normally being reduced relative to the lateral bending force required to fold the attachment ring 44 formed by a single turn of nitinol wire having a diameter of 0.010 in.

The vascular graft 10 may be treated with anti-thrombogenic agents (such as heparin, heparin derivatives, urokinase, and PPack (dextrophenylalanine proline arginine chloromethylketone)), anti-proliferative agents (such as enoxaprin, angiopeptin, or monoclonal antibodies capable of blocking smooth muscle cell proliferation, hirudin, and acetylsalicylic acid), anti-inflammatory agents (such as dexamethasone, prednisolone, corticosterone, budesonide, estrogen, sulfasalazine, and mesalamine), antineoplastic/antiproliferative/anti-miotic agents (such as paclitaxel, 5-fluorouracil, cisplatin, vinblastine, vincristine, epothilones, endostatin, angiostatin and thymidine kinase inhibitors), anesthetic agents (such as lidocaine, bupivacaine, and ropivacaine), anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides), vascular cell growth promotors (such as growth factor inhibitors, growth factor receptor antagonists, transcriptional activators, and translational promotors), vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin), cholesterol-lowering agents, vasodilating agents, and agents which interfere with endogenous vascoactive mechanisms.

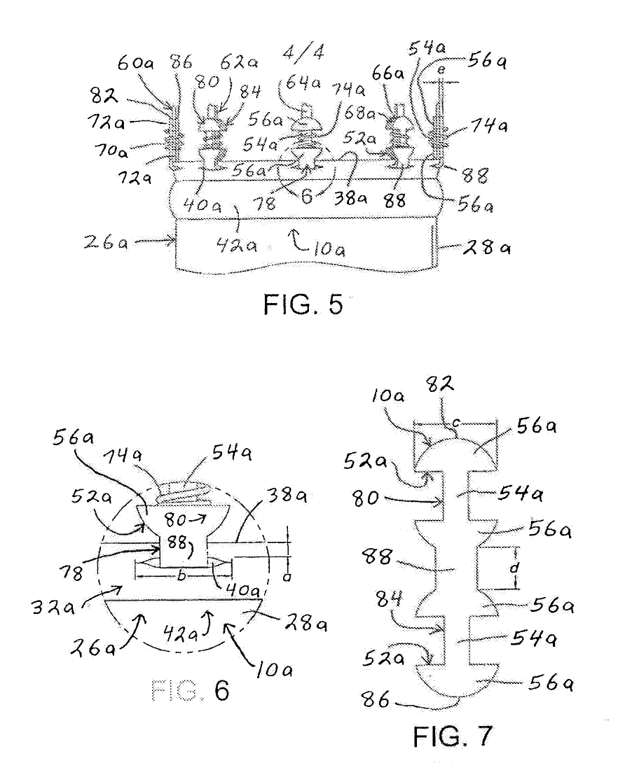

An alternative embodiment of the vascular graft 10a is shown in FIGS. 5 to 7. Parts illustrated in FIGS. 5 to 7 which correspond to parts illustrated in FIGS. 1 to 3 have, in FIGS. 5 to 7, the same reference numeral as in FIGS. 1 to 3 with the addition of the suffix “a”. In this alternative embodiment, the end surface 38a is defined by the edge of the tubular structure 26a included in the end structure 32a. The slits 40a are circumferential and located between the end surface 38a and cuff or sealing ring structure 42a. The longitudinal dimension a between the end surface 38a and each of the slits 40a, shown in FIG. 6, is between 1 mm. and 2 mm., and in a preferred embodiment, the longitudinal dimension a is 1.5 mm.

A preferred embodiment of the tubular structure 26a is formed of ePTFE having a node and fibril micro-structure in which the fibrils are oriented at between 30 degrees and 45 degrees relative to a transverse plane of the tubular structure 26a. The transverse plane of the tubular structure 26a is perpendicular to the longitudinal axis thereof. The 30 degrees to 45 degrees orientation refers to the fibrils of the node and fibril micro-structure. A further preferred embodiment of the tubular structure 26a is formed of ePTFE material having a node and fibril micro-structure in which the fibrils have a 30 degree orientation relative to the transverse plane.

A further preferred embodiment of the tubular structure 26a is formed of ePTFE material having a node and fibril micro-structure in which the fibrils have inclined orientations relative to the transverse plane and the inclined orientations oppose one another such as by having alternating inclinations or cross-directions. The inclined orientations of the fibrils which oppose one another may be provided by coaxial tubular structures 26a each having respective node and fibril micro-structures. The fibrils of the respective node and fibril micro-structures of the coaxial tubular structures 26a have corresponding inclinations relative to the transverse plane which are uniform in the respective tubular structures 26a. The respective inclinations of the fibrils in the corresponding tubular structures 26a are in opposite directions relative to the transverse plane to provide the end structure 32a with an ePTFE micro-structure having fibrils which have the opposed inclinations.

The vascular graft 10a includes strap structures 78 each of which extends through a respective slit 40a, as shown in FIGS. 5 and 6. The width c of each strap structure 70a, shown in FIG. 7, is less than the length b of each slit 40a, shown in FIG. 6, to allow the insertion of each strap structures 78 through the corresponding slit 40a.

The strap structures 78 each have an outer leg structure 80 which has an end surface 82. The strap structures 78 each have an inner leg structure 84 which has an end surface 86. The outer and inner leg structures 80, 84 are connected by an intermediate structure 88 which is located between the corresponding end surfaces 82, 86.

The outer and inner leg structures 80, 84 are each defined by corresponding dogbone structures 52a. The dogbone structures 52a have configurations which are substantially the same such that the folding of each strap structures 78 results in the dogbone structures 52a of the corresponding outer and inner leg structures 80, 84 being superimposed relative to one another, as shown in FIG. 5.

The strap structures 78 are located within the slits 40a such that the intermediate structures 80a have a transversely symmetrical orientation relative to the corresponding slit. The strap structures 78 are each folded such that the end surface 38a is located longitudinally between the slits 40a and the end surfaces 82, 86. Consequently, the folded strap structures 78 extend from the slits 40a in the proximal direction relative to the tubular structure 26a, as shown in FIG. 5. The longitudinal dimension a between the end surface 38a and each of the slits 40a, shown in FIG. 6, is related to the dimensions d, e shown in FIGS. 5 and 7 according to the following:

d>(2a)+e

where

e=thickness of strap structure 78

The locations of the strap structures 78 relative to the cross-section of the tubular structure 26a provides for each of the strap structures 78 to adjoin a corresponding dogbone structure 68a connected to the distal end 66a of the frame structure 62a. The dogbone structures 68a are each located between the outer and inner leg structures 80, 84 of a respective strap structures 78, as shown in FIG. 5. The configurations and sizes of the dogbone structures 68a are substantially the same as the configurations and sizes of the dogbone structures 52a. Consequently, each of the dogbone structures 68a is superimposed relative to the dogbone structures 52a of the outer and inner leg structures 80, 84 between which the dogbone structures 68a are located, as shown in FIG. 5.

The coil structure 74a is wound around the respective assemblies of the central members 54a and the adjoining central members 70a located between the central members 54a to secure the adjoining dogbone structures 52a, 60a together, as shown in FIG. 5. The ear members 56a, 72a obstruct relative longitudinal displacement between the adjoining dogbone structures 52a, 68a since such displacement results in the coil structures 74a engaging the ear members 66a, 72a. The connections between the respective pairs of dogbone structures 52a, 68a by the coil structure 74a provide for the connection of the tubular structure 26a to the stent attachment structure 60a. The stent attachment structure 60a extends from the tubular structure 26a in the proximal direction relative thereto.

The ePTFE material having a node and fibril micro-structure in which the fibrils are oriented between 30 degrees and 45 degrees relative to the transverse plane in the tubular structure 26a provides the end structure 32a with increased resistance to possible tearing thereof by the strap structure 78 which may be caused by longitudinal displacement thereof in the proximal direction relative to the tubular structure. The ePTFE material having a node and fibril micro-structure in which the fibrils have the 30 degree orientation in the tubular structure 26a provides the end structure 32a with increased resistance to possible tearing of the end structure 32a by the strap structures 78 being longitudinally displaced in the proximal direction relative to the tubular structure. The ePTFE material having a node and fibril micro-structure including fibrils which have the opposing inclinations in the tubular structure 26a provides the end structure 32a with increased resistance to tearing from longitudinal displacement of the strap structures 78 in the proximal direction relative to the tubular structure 26a.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

What is claimed is:

1. A vascular graft for location within a lumen of a vessel, said vascular graft comprising:
a tubular structure having an end structure;
an attachment ring connected to said end structure, said attachment ring being contained in a ring plane which is perpendicular to said end structure; and
a stent attachment structure connected to said attachment ring, said stent attachment structure having an anchor structure for connection to an inner surface of the vessel to secure the location of said vascular graft within the lumen.

2. A vascular graft according to claim 1, wherein said stent attachment structure extends from said attachment ring in a proximal direction relative to said tubular structure.

3. A vascular graft according to claim 1, wherein said end structure has a transverse end surface,
said attachment ring comprising a ring structure connected to said end structure, said ring structure being contained in said ring plane,
said attachment ring comprising one or more loop structures integrally connected to said ring structure, said one or more loop structures extending from said ring structure in a proximal direction relative to said tubular structure,
said stent attachment structure being connected to said one or more loop structures for said connection of said stent attachment structure to said attachment ring.

4. A vascular graft according to claim 1, wherein said attachment ring comprises a wire structure having a plurality of wind structures.

5. A vascular graft according to claim 1, wherein said end structure comprises an outer layer and an inner layer between which said attachment ring is secured to provide said connection of said attachment ring to said end structure.

6. A vascular graft according to claim 5, wherein said end structure has a transverse end surface,
said end surface being defined by an integral junction of said outer layer and inner layer, said end structure comprising one or more slits formed in said end surface,
said stent attachment structure comprising one or more stent structures each of which extends through a respective one of said one or more slits to said attachment ring for connection thereto to provide said connection of said stent attachment structure to said attachment ring.

7. A vascular graft according to claim 1, wherein said stent attachment structure has an eyelet through which said attachment ring is threaded to provide said connection of said stent attachment structure thereto.

8. A vascular graft according to claim 1, wherein said stent attachment structure has an eyelet,
said vascular graft comprising a coil structure which is threaded through said eyelet, said coil structure encircling said attachment ring to provide said connection of said stent attachment structure thereto.

9. A vascular graft according to claim 1, wherein said end structure has a transverse end surface,
said vascular graft further comprising a cuff or sealing ring structure which is secured to said end structure such that said attachment ring structure is located longitudinally between said cuff or sealing ring structure and end surface.

10. A vascular graft according to claim 9, wherein said cuff or sealing ring structure is inflatable.

11. An attachment ring for connection to a vascular graft, said attachment ring comprising:
a circular ring structure for connection to the vascular graft, said ring structure being contained in a ring plane;
one or more loop structures integrally connected to said ring structure, said one or more loop structures each being contained in a respective loop plane, each of said one or more loop planes being perpendicular to said ring plane, said loop structures each extending from said ring structure in respective directions which are the same.

12. An attachment ring according to claim 11, wherein said ring structure comprises a wire structure having a plurality of wind structures, said wind structures extending continuously into said loop planes to provide said loop structures.

13. A vascular graft for location within a lumen of a vessel, said vascular graft comprising:
a tubular structure having an end structure, said tubular structure having one or more circumferential slits formed in said end structure;
one or more strap structures each of which extends through a respective one of said one or more circumferential slits and is folded such that said one or more strap structures each has an outer leg structure and an inner leg structure; and
a stent attachment structure comprising one or more stent structures each of which is connected to said outer leg structure and inner leg structure of a respective one of said one or more strap structures, said one or more stent structures each having an anchor structure for connection to an inner surface of the vessel to secure the location of said vascular graft within the lumen.

14. A vascular graft according to claim 13, wherein said end structure has a transverse end surface,
said outer leg structures and inner leg structures each having respective end surfaces,
said folding of said one or more strap structures providing for said end surface of said end structure to be located longitudinally between said one or more circumferential slits and said end surfaces of said outer leg structure and inner leg structure,
said connection of said one or more stent structures to respective ones of said outer leg structure and inner leg structure providing for said one or more stent structures being located between said respective ones of said outer leg structure and inner leg structure.

15. A vascular graft according to claim 14, wherein said one or more stent structures each have a dogbone structure,
said end structure of each said outer leg structure and inner leg structure having a dogbone structure which corresponds to said dogbone structures of said one or more stent structures,
said connection of said one or more stent structures to respective ones of said one or more strap structures providing for said dogbone structures of said one or more stent structures being located between respective ones of said dogbone structures of said outer leg structure and inner leg structure such that said dogbone structures of said one or more stent structures and respective ones of said one or more strap structures are superimposed relative to one another,
said vascular graft comprising one or more connector structures encircling said superimposed dogbone structures of each of said one or more stent structures and respective one of said one or more strap structures to secure said superimposed dogbone structures to one another.

16. A vascular graft according to claim 15, wherein said one or more connector structures each comprise a coil structure.

17. A vascular graft according to claim 13, wherein said stent attachment structure extends from said one or more strap structures in a proximal direction relative to said tubular structure.

18. A vascular graft according to claim 13, wherein said end structure comprises ePTFE having a node and fibril micro-structure including fibrils which have an inclined orientation relative to a transverse plane of said tubular structure.

19. A vascular graft according to claim 13, wherein said end structure has a transverse end surface,
said vascular graft further comprising a cuff or sealing ring structure which is secured to said end structure such that said circumferential slits are located longitudinally between said cuff or sealing ring structure and end surface.

20. A vascular graft according to claim 19, wherein said cuff or sealing ring structure is inflatable.

21. A method of making a vascular graft comprising:
providing a tubular structure having an end edge and one or more circumferential slits;
providing an attachment ring contained in a ring plane;
locating the attachment ring to encircle the tubular structure;
folding outward and backward a longitudinal portion of the tubular structure between the end edge and attachment ring such that the attachment ring is located between the portion of the tubular structure which is folded outward and backward and the longitudinal portion of the tubular structure within the portion of the tubular structure which is folded outward and backward, the longitudinal portion of the tubular structure which is folded outward and backward defining an outer layer of the tubular structure, the longitudinal portion of the tubular structure which is within the outer layer defining an inner layer of the tubular structure such that the attachment ring is located between the inner layer and outer layer, the outer layer and inner layer being joined by an integral junction which defines a transverse end surface of the tubular structure;
locating the outer layer longitudinally relative to the inner layer such that the one or more circumferential slits are located on the end surface;
securing the outer layer to the inner layer to connect the attachment ring to the tubular structure; and
connecting the attachment ring to a stent attachment structure through the one or more circumferential slits, the stent attachment structure having an anchor structure for connection to an inner surface of a vessel to secure the location of the vascular graft within a lumen of the vessel.

22. A method of making a vascular graft comprising:
providing a tubular structure having one or more circumferential slits;
inserting a respective strap structure through each of the one or more circumferential slits;
folding each of the one or more strap structures such that the one or more strap structures each have an outer leg and an inner leg; and
connecting each of the outer leg structures and inner leg structures to a stent attachment structure, the stent attachment structure having an anchor structure for connection to an inner surface of a vessel to secure the location of the vascular graft within a lumen of the vessel.
